(19) United States
(12) Patent Application Publication
(10) Pub. No.: US 2008/0044890 A1
(43) Pub. Date: Feb. 21, 2008

(54) Interchangable sleeve for enhancing proliferation of cells in a rotating bioreactor

(76) Inventor: Clayton R. Parker, Safety Harbor, FL (US)

Correspondence Address: LADAS & PARRY LLP, 224 SOUTH MICHIGAN AVENUE, SUITE 1600, CHICAGO, IL, 60604, US

(21) Appl. No.: 11/169,386

(22) Filed: Jun. 29, 2005

Related U.S. Application Data
(60) Provisional application No. 60/584,506, filed on Jun. 30, 2004.

(51) Int. Cl. C12M 1/42

(52) U.S. Cl. 435/286.1; 435/298.2

(57) ABSTRACT

An interchangeable sleeve for enhancing proliferation of cells in a rotating bioreactor is disclosed with the interchangeable sleeve having a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve.

### BACKGROUND OF THE INVENTION

1. Field of the Invention

The present invention relates generally to a device for enhancing proliferation of cell cultures in a rotating bioreactor. Specifically, this invention relates to an interchangeable sleeve that encompasses a rotating bioreactor with the sleeve supplying a time varying electromagnetic force to the culture chamber of the bioreactor in order to increase cell growth and proliferation within the rotating bioreactor. More specifically, the present invention relates to an interchangeable sleeve that can encompass a rotating substantially cylindrical bioreactor and supply a time varying electromagnetic force of from 0.05 gauss to 0.5 gauss to the bioreactor chamber to substantially increase cell growth and proliferation within the bioreactor culture chamber.

The preferred embodiment is an interchangeable sleeve for enhancing proliferation of cells in a rotating bioreactor, with the sleeve comprising: a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve; and means for applying a pulsating time varying electromagnetic current to the electrical conductive material to create the time varying electromagnetic force of from approximately 0.05 gauss to 0.5 gauss within the cylindrical portion of the sleeve.

2. Description of the Prior Art

Wolf and Goodwin, in U.S. Pat. No. 6,673,597, patented the use of a time varying electromagnetic force to develop and proliferate cell cultures. Commercial utilization of this technology has provided two approaches to bioreactor design. The first approach is the use of baffles or plates within the bioreactor culture chamber with a time varying electromagnetic current applied across the plates to induce a time varying electromagnetic force within the culture chamber. The second approach is the use of a coil wrapped around the rotating bioreactor chamber and affixed thereto with a time varying electromagnetic current applied to the coil to create a time varying electromagnetic force within the culture chamber.

The problem with the prior art designs for application of a time varying electromagnetic force (TVEMF) to a rotating bioreactor chamber is that the coil used to induce the TVEMF or the plates within the bioreactor are part of the bioreactor chamber. Since the goal of proliferation of cell cultures is in many instances the utilization of the cell cultures for reintroduction into the human body for tissue regeneration or treatment of human maladies, the bioreactor chamber must meet the rigid standards of the Food and Drug Administration. Consequently, rather than comply with the rigid requirements the Food and Drug Administration for cleaning the bioreactor chamber to guarantee there is no contamination of the cell culture within the chamber, it would be highly desirable to have a disposable culture chamber. With a disposable chamber, the culture chamber could be manufactured pursuant to the rigid requirements the Food and Drug Administration and packaged in a sterile environment and container thereby enabling it to be used by the medical or research professional much the same as other disposable medical devices are used. However, if the TVEMF inducing device is incorporated into the disposable culture chamber, it significantly complicates the manufacture and sterilization process, and it requires that the TVEMF inducing device be disposed of along with the discarding of a used bioreactor chamber thereby significantly adding to the cost of the equipment and culturing process.

It would be highly desirable to provide disposable bioreactor culture chambers without the TVEMF inducing device being an integral part thereof.

The present invention overcomes problems, associated with prior art bioreactor designs by allowing disposable bioreactor culture chambers to be provided without the need to dispose of the bioreactor chamber TVEMF inducing device.

### SUMMARY OF THE INVENTION

The present invention relates to an interchangeable sleeve for enhancing proliferation of cells in a rotating bioreactor. The interchangeable sleeve is used to encompass a horizontal, cylindrical rotating bioreactor culture chamber and is designed to impart a time varying electromagnetic force of from 0.05 gauss to 0.5 gauss to the bioreactor culture chamber.

The interchangeable sleeve of this invention is an independent time varying electromagnetic wave generating coil that can be used with a disposable bioreactor culture chamber to significantly increase cell growth and proliferation in the culture chamber.

It is an object of this invention to provide an interchangeable sleeve for use with a cylindrical rotating bioreactor chamber.

It is a further object of this invention to provide an interchangeable sleeve for use with a cylindrical rotating bioreactor chamber wherein the interchangeable sleeve imparts a time varying electromagnetic force (square wave, Fourier curve) to the bioreactor chamber.

Other aspects, features and advantages of the present invention will be apparent from the following description of the presently preferred embodiments of the invention given for the purpose of disclosure.

### BRIEF DESCRIPTION OF THE DRAWINGS

In the drawings,

FIG. 1 is a side view of the bioreactor improvement used in this invention;

FIG. 2 is a perspective view of the bioreactor improvement used in this invention;

FIG. 3 is a side view showing the assembly of the bioreactor improvement of this invention; and

FIG. 4 is a side view showing the assembly of the bioreactor improvement of this invention.

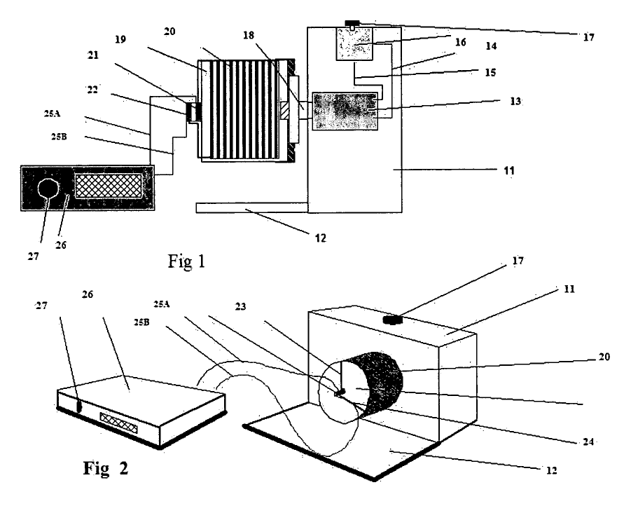

### DETAILED DESCRIPTION OF THE INVENTION

In the drawings, a motor housing 11 is supported by a base 12. A motor 13 is attached inside the motor housing 11 and connected by wires 14 and 15 to a control box 16 that has a control mechanism therein such that the speed of the motor can be incrementally controlled by turning the control knob 17. The motor housing 11 has a motor 13 inside set so that the motor shaft 18 extends through the housing with the motor shaft 18 being longitudinal, that is, so that the center of the shaft is parallel to the plane of the earth at the location of the bioreactor. A longitudinal cylinder 19 is connected to the shaft so that the cylinder rotates about its longitudinal axis with the longitudinal axis parallel to the plane of the earth. The cylinder is wound on its outside with a wire coil 20. The size of the wire and number of times it is wound around the cylinder are such that when a square wave current of from 0.1 mA to 1000 mA is supplied to the wire coil, an electromagnetic field of from 0.05 gauss to 6 gauss is generated within the cylinder. The wire coil 20 is connected to rings 21 and 22 at the and of the shaft by wires 23 and 24. These rings are then contacted by wires 25A and 25B in such a manner that the cylinder can rotate while the current is constantly supplied to the coil. An electromagnetic generating device 26 is connected to the wires 25A and 25B. The electromagnetic generating device supplies a square wave to the wires and coil by adjusting its output by turning the knob 27.

In operation, the cylinder is opened and the cell culture placed therein. The speed of the cylinder is adjusted visually so that the cell culture substantially remains at or about the longitudinal axis of the cylinder. The electromagnetic generating device is turned on and adjusted so that the square wave output generates the desired electromagnetic field in the cylinder, from 0.05 gauss to 6 gauss. The electromagnetic field can be measured by standard equipment such as an EN131 CellSensor Gauss Meter available at electronic stores or on the Internet by placing the meter next to the cylinder.

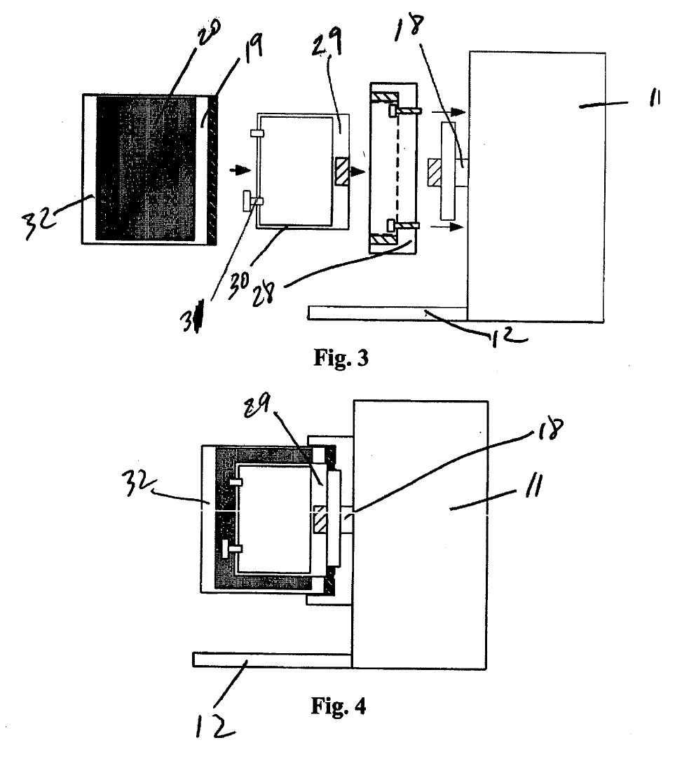

FIG. 3 and FIG. 4 show the disposable sleeve and its assembly. A rotatable mounting 28 on the bioreactor holds the disposable culture chamber 29. The disposable culture chamber 29 is attached to the bioreactor culture chamber 30 by a screw 31. The time varying electromagnetic force apparatus 32 is a coil 20 wound on a cylinder 19 and is inserted over the culture chamber. Since the time varying electromagnetic force apparatus 32 is independent of the culture chamber 30, it can be removed and the culture chamber 30 discarded so the time varying electromagnetic force apparatus 32 can be reused. The time varying electromagnetic force apparatus 32 must generate a time varying electromagnetic force of from 0.05 gauss to 0.5 gauss and this is generally accomplished by approximately ten windings per inch.

In application, collected peripheral blood cells PBCs (0.75×10⁶ cells/ml) obtained from donors are suspended in Iscove's modified Dulbecco's medium (IMDM) (GIBCO, Grand Island, N.Y.) supplemented with 5% human albumin (HA) or 20% human plasma, 100 ng/ml recombinant human G-CSF (Amgen Inc., Thousand Oaks, Calif.), and 100 ng/ml recombinant human stem cell factor (SCF) (Amgen). D-Penicillamine [D(−)-2-Amino-3-mercapto-3-methylbutanoic acid] (Sigma-Aldrich) a copper chelation agent, is dissolved in DMSO. 10 ppm of the D-Penicillamine is introduced into the cell mixture. One sample of the culture mix is placed into the rotatable bioreactor described herein. A time varying electromagnetic force of approximately 0.5 gauss was created in the cylinder that was over the rotatable bioreactor. A second sample was placed in a rotatable bioreactor without any time varying electromagnetic force applied thereto.

After the seventh day of expansion, the cells were washed with PBS and analyzed by conventional counting techniques, for example by using a Coulter counter. The sample exposed to the time varying electromagnetic force had more than five times the growth or expansion of the sample that was not exposed to the time varying electromagnetic force.

Having fully described this new and unique invention,

I claim:

1. An interchangeable sleeve for enhancing proliferation of cells in a rotating bioreactor, said sleeve comprising:
a. a substantially cylindrical and substantially rigid electrical conductive material wound in a cylindrical shape and capable of being connected to a pulsating time varying electromagnetic current to create a time varying electromagnetic force of from approximately 0.05 gauss to 0.5 gauss within the cylindrical portion of the sleeve; and
b. means for applying a pulsating time varying electromagnetic current to the electrical conductive material to create the time varying electromagnetic force of from approximately 0.05 gauss to 0.5 gauss within the cylindrical portion of the sleeve.

2. An interchangeable sleeve as in claim 1 wherein the electrical conductive material is electrical conductive wire.

3. An interchangeable sleeve as in claim 1 wherein the time varying electromagnetic force is a square wave.

4. An interchangeable sleeve as in claim 2 wherein the electrical conductive wire is wound in the cylindrical shape at approximately ten windings per inch.

5. An interchangeable sleeve as in claim 1 wherein the substantially cylindrical and substantially rigid electrical conductive material wound in a cylindrical shape is electrical conductive wire wound about an electromagnetically permeable polymer with a substantially cylindrical shape.

6. An interchangeable sleeve as in claim 5 wherein the electrical conductive wire is wound in the cylindrical shape at approximately ten windings per inch.

7. An interchangeable sleeve as in claim 5 wherein the electrical conductive wire is a ferromagnetic metal.

8. An interchangeable sleeve as in claim 5 wherein the sleeve has means for attaching it to the bioreactor in a manner that allows the bioreactor culture chamber to rotate within the sleeve.

9. An interchangeable sleeve as in claim 5 wherein the sleeve has means for attaching it to the bioreactor culture chamber to allow it to rotate with the culture chamber.

10. An apparatus for growing cells, said apparatus comprising a cylindrical rotating culture chamber, means for rotating the culture chamber at a rotational speed that prevents cells therein from substantially contacting the sides of the chamber, and an interchangeable sleeve encompassing the cylindrical portion of the culture chamber and being capable of supplying a time varying electromagnetic force of from approximately 0.05 gauss to 0.5 gauss to the internal portion of the culture chamber while it rotates.

11. An apparatus as in claim 10 wherein the interchangeable sleeve has means for controlling the temperature of the culture chamber.

12. An apparatus as in claim 10 wherein the time varying electromagnetic force is a square wave.
